(19) United States
(12) Patent Application Publication
(10) Pub. No.: US 2006/0246146 A1
(43) Pub. Date: Nov. 2, 2006

(54) Method of increasing the salivary sialic acid content in a mammal

(76) Inventors: Robert J. McMahon, Evansville, IN (US); Bing Wang, Wolli Creek (AU); Jennie Brand-Miller, Greenwich (AU)

Correspondence Address: NELSON MULLINS RILEY & SCARBOROUGH, LLP, MERIDIAN, SUITE 1700, 1320 MAIN STREET, COLUMBIA, SC, 29201, US

(21) Appl. No.: 11/118,845

(22) Filed: Apr. 29, 2005

(51) Int. Cl. A61K 38/38; A61K 35/20; A61K 36/48

(52) U.S. Cl. 424/535; 514/8; 424/757

(57) ABSTRACT

A method of increasing the salivary sialic acid content of a mammal is described which involves administering to the mammal casein glycomacropeptide in an amount sufficient to increase the salivary sialic acid content of the mammal.

### BACKGROUND OF THE INVENTION

(1) Field of the Invention

The present invention relates to a method of increasing the salivary sialic acid content in a mammal, and more particularly to a method of increasing the salivary sialic acid content in a mammal by administration of a dietary source of sialic acid.

(2) Description of the Related Art

It is known that sialic acid is an important recognition molecule for specific pathogenic organisms that use it to selectively bind to the epithelial layer of the host for colonization. See Alexander, D. A. et al., J. Virol., 76:11265-11272 (2002), Arnberg, N. et al., J. Virol., 74:42-48 (2000), Arnberg, N., et al., Virology, 302:33-43 (2002), Arnberg, N., et al., J. Virol., 76:8834-8841 (2002), Chen, M. H. et al., Virology, 233:440-442 (1997), Ciarlet, M., et al., J. Viroll, 76:4087-4095 (2002), Connolly, J. L., et al., J. Virol., 75:4029-4039 (2001), Dormitzer, P. R., et al., J. Virol. 76:10512-10517 (2002), Krempl, C., et al., J. Gen. Virol., 81:489-496 (2000), Kuhlenschmidt, T. B., et al., Adv Exp Med Biol, 473: 309-317 (1999), Miller-Podraza, H., et al., Infect Immun 67: 6309-6313 (1999), Schwegmann, C., et al., Virus Res 75: 69-73 (2001), Shah, A. H., et al., Virology 304: 443-450 (2002), and Takahashi, Y., et al., Infect. Immun., 70:1209-1218 (2002). It is also known that the saliva of breastfed infants is significantly higher in sialic acid than that of formula fed infants, which may possibly be related to the decreased sialic acid intake and salivary sialic acid in formula fed infants (Carlson, S. E., Am. J. Clin. Nutr., 41:720-726 (1985), Tram, T. H., et al., Arch. Dis. Child, 77:315-318 (1997), and Wang, B., et al., J. Pediatr., 138:914-916 (2001)).

Human breastmilk contains substantial amounts of sialic acid bound to glycoproteins, oligosaccharides, and lipids (Carlson (1985), id., and Wang, B. et al., J. Pediatr., 74:510-515 (2001). Infant formula also contains sialic acid bound to these components, but in a very different distribution (Martin et al. (2001), Id., Sanchez-Diaz, et al., (1997) Id., and Wang et al., (2001a), Id.). In addition to the difference in distribution, infant formula contains substantially less total sialic acid compared to early human breastmilk. Carlson (1985), Id., has reported that, although pooled samples of human breastmilk show sialic acid levels of around 400 mg/L, samples of milk from mother's immediately after parturition can be as high as 1500 mg/L, or more.

Cow's milk-based formulas generally have low sialic acid content. In one study, the concentration of sialic acid in several casein/whey combination formulas was less than 200 mg sialic acid/L. Moreover, soy protein-based formulas contain substantially reduced levels of sialic acid as compared to cow's milk-based formula. Therefore, formulas that are both lactose free and soy protein-based would exhibit very low sialic acid content. In some instances, research has shown that dietary supplementation with sialic acid, or with a sialic acid containing material provides certain benefits.

There are several known sources of sialic acid in its various conjugated forms. These include, but are not limited to, free N-acetylneuraminic acid (or sialic acid), the oligosaccharide sialyllactose, sialic acid-containing gangliosides, and the protein casein macropeptide (CMP), the major portion of which is glycomacropeptide (GMP), and, when obtained from cow's milk, this is referred to as casein glycomacropeptide (CGMP).

A method of producing CGMP is described in U.S. Patent Application 20040022918, which teaches that the manufacture of cheese from milk by coagulating cow's milk with rennet causes the coagulum to contract into a curd as it expresses whey. Casein macropeptide (CMP) is cleaved from the casein protein as a result of the action of the rennet on kappa casein and about 90% of the CMP is typically removed with the whey. CMP is a heterogeneous group of proteins, which contain all the genetic variations and post-translational modifications of kappa casein (Yvon et al., Reprod. Nutr. Dev., 34:527-537 (1994)). The predominant carbohydrate is sialic acid. Glycomacropeptide or GMP is the principal (50 to 75%) component of CMP. The carbohydrate content of the GMP renders it soluble in a 12% trichloroacetic acid solution. A number of analytical measurement techniques include a pre-treatment, which involves a TCA solution, this may remove at least a portion of the non-glycosylated CMP. For example the method published in The Official Journal of the European Communities (L228/10 Annex IV), details a HPLC method for measuring GMP in dairy products and uses the GMP level to calculate the level of cheese whey present in a sample. Other methods of producing CGMP from milk are described by Brody, E. P., in Br. J. of Nutr., 84(Suppl. 1):S39-S46 (2000), and in PCT/US94/15952, WO 03/049547, and in U.S. Pat. Nos. 6,555,659, 5,280,107, 5,968,586, and 5,075,424, and in U.S. Patent Application Publication No. 2002/0183489.

The addition of sialic acid or sources of sialic acid to certain nutritional formulas has been discussed in U.S. Pat. No. 6,506,422, which discloses a particular nutritional formula containing casein glycomacropeptide and complimentary essential amino acids other than phenylalanine for administration to patients suffering from phenylketonuria. The levels of sialic acid found in infant formulas are not mentioned.

Furthermore, several patents appear to describe nutritional formulations for the inhibition of the binding of pathogenic organisms. See, for example EP 0904784, WO 2004/002495, and U.S. Pat. Nos. 5,505,955, 5,576,300, 6,630,452, 5,670,201, and U.S. Patent Publication No. 2003/0129278. However, at least in U.S. Pat. No. 5,576,300 to Mukerji, et al., it was found that sialic acid residues in bovine kappa-casein were not required for the inhibitory effect against infection by human rotavirus.

Kelleher, S. L., et al., in Am J. Clin. Nutr., 77(5):1261-1268 (2003), described supplementation of the diet of infant rhesus monkeys with glycomacropeptide and found that such supplementation increased zinc absorption, but did not discuss either its effect on salivary sialic acid, or its effect on inhibition to infection by pathogenic organisms.

Several patents discuss the probiotic affects of dietary sialic acid (See, e.g., U.S. Pat. Nos. 4,963,384, 6,630,452, and 4,925,680), but dietary supplementation with casein glycomacropeptide is not discussed.

U.S. Pat. No. 6,270,827, discloses a formulation containing human milk proteins or recombinant host resistance factors, one of which is recombinant human kappa-casein, to supplement synthetic infant formulas.

U.S. Pat. No. 4,762,822 discloses the use of N-acetylneuraminic acid or gangliosides containing sialic acid in infant formula to protect the newborn from gastrointestinal disease-producing organisms.

International patent application WO 01/60346 A2 discloses a nutritional formulation containing the oligosaccharides oligofructose and sialyllactose as prebiotic substances to promote the growth of bifidobacteria in the gut that may be used in conjunction with infant formula.

WO-A-00 49885 describes the use of a milk protein hydrolysate for addressing bone or dental disorders. Casein glycomacropeptide (CGMP) is extracted from sweet whey by a combination of electrodialysis, cation exchange resin, anion exchange resin, evaporation, spray drying, ultrafiltration and freeze drying, and is used to enrich foods or liquid enteral compositions.

Although the dietary administration of sialic acid has been reported for several purposes, it has not been shown to cause an increase in salivary sialic acid content in an animal model of infant nutrition and development.

Because dietary supplementation is an easy and widely accepted method of administering various agents to subjects, and in particular to infant mammals, it would be useful to provide a method by which the salivary sialic acid content in a mammal could be increased by dietary means. Such a method would be particularly useful for neonatal mammal subjects that were in need of increasing salivary sialic acid content.

### SUMMARY OF THE INVENTION

Briefly, therefore the present invention is directed to a novel method of increasing the salivary sialic acid content in a mammal, the method comprising administering to the mammal casein glycomacropeptide in an amount sufficient to increase the salivary sialic acid content in the mammal.

The present invention is also directed to a novel method of increasing salivary sialic acid content in a mammal, the method comprising: determining whether the mammal is one that is in need of increasing salivary sialic acid content; and, if so, administering to the mammal a formula comprising casein glycomacropeptide in an amount sufficient to increase salivary sialic acid content in the mammal.

Among the several advantages found to be achieved by the present invention, therefore, may be noted the provision of a method by which the salivary sialic acid content in a mammal are increased by dietary means. Such a method would be particularly useful for neonatal mammal subjects that were in need of an increase in salivary sialic acid content.

### BRIEF DESCRIPTION OF THE DRAWINGS

FIG. 1 is a plot of the mean salivary sialic acid content of piglets in control and treatment groups versus the age of the animals in days, where the piglets in the control group received a standard diet of soy/whey/casein sow milk pig-replacer containing 150 mg/L of naturally occurring sialic acid, and piglets in the treatment group received a similar formula wherein casein glycomacropeptide replaced enough of the casein that the level of sialic acid in the diet was 600 mg/L, where the plot shows the higher salivary sialic acid level in piglets receiving the dietary casein glycomacropeptide.

### DETAILED DESCRIPTION OF THE PREFERRED EMBODIMENTS

In accordance with the present invention, it has been discovered that the salivary sialic acid content in a mammal can be increased by administering to the mammal casein glycomacropeptide in an amount sufficient to increase the salivary sialic acid content in the mammal. The new method has been found to be particularly useful when the mammal is one in which an increase in salivary sialic acid content is needed.

In an alternative embodiment, the present method comprises determining whether the mammal is one that is in need of increasing salivary sialic acid content; and, if so, administering to the mammal a formula comprising casein glycomacropeptide in an amount sufficient to increase salivary sialic acid content in the mammal.

The inventors have shown that in piglets, which are a well known and widely used model for infant development and nutrition, feeding with a nutritional enteral formulation that is supplemented with an amount of casein glycomacropeptide sufficient to provide a total sialic acid content of 600 mg/L significantly increased the salivary sialic acid content over feeding with the same diet without CGMP supplementation. The inventors believe that such an increase in the salivary sialic acid content of a mammal is an important component in improving the efficacy of the protective secretions in the nasopharyngeal region in relation to the inhibition of binding of pathogenic organisms.

The inventors previously showed that preterm infants fed human milk had twice the level of salivary sialic acid as infants fed commercial formula, and that this increased level of sialic acid suggested greater viscosity and enhanced protection of the mucosal surfaces. See Wang, B. et al., J. Pediatr., 138(6):914-916 (2001), and Tram, T. H. et al., Arch. of Disease in Childhood, 77:315-318 (1997). It would be expected, therefore, that the increase in salivary sialic acid caused by the present invention would have the same advantageous effect as that shown in the earlier work with human milk—namely, that the increase in salivary sialic acid due to the administration of CGMP would increase saliva viscosity and enhance protection of mucosal surfaces.

The inventors have found that casein glycomacropeptide is a surprisingly good source for dietary supplementation to raise the sialic acid content of saliva. This is because cow's milk, in general, is low in sialic acid, and also, the macromolecules to which sialic acid is bound are different than those found in human milk, for example. Nonetheless, the inventors have found casein glycomacropeptide to be a surprisingly effective dietary supplement to raise salivary sialic acid content. Furthermore, CGMP is relatively inexpensive, readily available, and nutritionally safe for infants.

The present invention is useful for any mammal. However, it is particularly useful for humans. The mammal can be of any age. However, it has been found that the present method is particularly useful when the mammal is between about 1 day and about 4 years of age. This age range is meant to include infants and toddlers when the mammal is a human. It is preferred that the mammal is a neonatal mammal. As used herein, the term “neonatal” is meant to describe the ages between birth and about two years.

The present method is particularly useful when the mammal is one that is in need of increasing salivary sialic acid content. When the terms “in need of increasing salivary sialic acid content” are used herein, they are meant to describe a subject who could benefit, no matter how greatly or slightly, from an increase in the content of sialic acid in its saliva. As briefly discussed above, the present method optionally includes the step of determining whether the mammal is one that is in need of an increase in salivary sialic acid content, and, if so administering to the mammal a formula comprising casein glycomacropeptide in an effective amount.

The terms, “increase in salivary sialic acid content”, are meant to include any increase, no matter how small, in the content of sialic acid in the saliva of the subject mammal. The content of sialic acid in the saliva of the mammal can be measured by any one or more of several tests that are useful for measuring sialic acid in liquid biological samples. Examples of such tests include, without limitation, those described by Wang, B. et al., in Comp. Biochem. Phsiol, 119A(1):435-439 (1998), by Carlson, S., in Am. J. of Clin. Nutr., 41:720-726 (1985), and by Warren, L., in J. Biol. Chem., 234:1971-1975 (1959). In the present invention, it is preferred that the terms “salivary sialic acid content” refer to the content of sialic acid in saliva free of any sialic acid that is contributed directly to the saliva from an ingested nutrient. In other words, it is preferred that the content of sialic acid in saliva, as those terms are used herein, is the same as the sialic acid content in saliva as it issues from a saliva gland and before it has absorbed any sialic acid that might be present in the mouth or GI tract due to ingestion.

It is believed that a need of an increase in salivary sialic acid content can be caused in a mammal, and in a human in particular, by any of a number of factors that are known in the art. By way of example, malnutrition, the presence of environmental factors—such as ingestion of certain metals, deprivation of oxygen, trauma, disease, and other factors, can cause a need for such an increase. Accordingly, it is believed that in the present invention the need of an increase in salivary sialic acid content is shown, or the determination can be made that the mammal is one that is in need of an increase in salivary sialic acid content, where at least a portion of the mammal's nutritional requirement has been supplied by a formula having less than about 100 mg/L of sialic acid, and in particular, where substantially all of the mammal's nutritional requirement has been supplied by administration of a formula having less than about 100 mg/L of sialic acid.

When it is said that “at least a portion of the mammal's nutritional requirement has been supplied” by a certain formula, it is meant that at least 25% of the mammal's nutritional requirement has been supplied for at least a majority of the period from birth to the present age of the mammal by the formula. It is preferred that the portion of the mammal's nutritional requirement that has been supplied for at least a majority of the period from birth to the present age of the mammal is at least 50%, more preferred is 75%, and yet more preferred is substantially all of the mammal's nutritional requirement.

Due to the low level of sialic acid in soy protein and cow's milk protein, as compared with the sialic acid levels in human milk—especially in colostrum and milk produced during early lactation, it is believed that a mammal receiving a formula wherein the major part of the protein is soy protein or cow's milk protein, and/or in which the level of sialic acid is less than about 100 mg/L, is one that is in need of an increase in salivary sialic acid content. In particular, this is believed to be the case when at least about 75% by weight of the protein contained in the formula is soy protein or cow's milk protein, and is yet is more true when substantially all of the protein contained in the formula is soy protein or cow's milk protein.

In fact, it is believed that the need of an increase in salivary sialic acid content in a mammal is shown where the mammal's nutritional requirement has been supplied by a diet providing sialic acid in an amount that is lower than would normally be obtained from breastfeeding. When it is said that “the mammal's nutritional requirement has been supplied”, it is meant that the mammal's nutritional requirement has been supplied for at least a majority of the period from birth to the present age of the mammal. The terms “a diet providing sialic acid in an amount that is lower than would normally be obtained from breastfeeding”, means a diet comprising a liquid formula having a sialic acid content that is below about 100 mg/L. In other embodiments, this means a liquid formula having a sialic acid content that is below about 200 mg/L, and in other embodiments, a liquid formula having a sialic acid content that is below about 300 mg/L.

In the present invention, the casein glycomacropeptide, or formula that comprises casein glycomacropeptide, can be administered to the mammal by any type of enteral administration. As used herein, enteral administration includes administration of a formula to any point in the GI tract of the mammal, and includes without limitation, oral administration, and enteral tubular administration.

A formula that comprises casein glycomacropeptide can be any nutritional formula, but is preferably an infant formula. In some embodiments, the infant formula is a nutritionally complete infant formula comprising carbohydrate, lipid, and protein. The infant formula for use in the present invention can be nutritionally complete, or it can be a supplemental formula. Typically, the formula contains suitable types and amounts of lipids, carbohydrates, proteins, vitamins and minerals. The amount of lipids or fats typically can vary from about 3 to about 7 g/100 kcal. The amount of proteins typically can vary from about 1 to about 5 g/100 kcal. The amount of carbohydrates typically can vary from about 6 to about 16 g/100 kcal.

As used herein, the term “formula” means a man-made composition, and is not to be interpreted to include breast milk, for example.

Protein sources can be any used in the art, and may include, for example, nonfat milk, whey protein, casein, soy protein, hydrolyzed protein, and amino acids. Lipid sources can be any used in the art such as, for example, vegetable oils such as palm oil, soybean oil, palm olein oil, corn oil, canola oil, coconut oil, medium chain triglyceride oils, high oleic sunflower oil, and high oleic safflower oil. Carbohydrate sources can be any known in the art such as, for example, lactose, glucose polymers, corn syrup solids, maltodextrins, sucrose, starch, and rice syrup solids.

Conveniently, several commercially available infant formulas can be used as the basic formula for the CGMP additions. For example, Enfamil® Lipil with iron (available from Mead Johnson & Company, Evansville, Ind., U.S.A.) may be supplemented with an effective amount of CGMP and used to practice the method of the present invention. Particular infant formulas suitable for use in the present invention are described in the Examples herein.

The total protein in the formulation from all protein sources should be nutritionally appropriate for infants, which is typically from about 12 g per liter to 18 g per liter and, in some embodiments, may be about 14 g per liter. The total sialic acid in the formulation may be between about 200 and about 1500 mg per liter. It is preferred that the present formula comprises a liquid having a sialic acid concentration of at least about 200 mg/liter. In some embodiments, it is more preferred that the formula comprises a liquid having a sialic acid level of at least about 300 mg/liter, and a sialic acid level of at least about 600 mg/liter is yet more preferred. It is preferred that the formula contains up to 6 g per liter of casein glycomacropeptide (CGMP) or related protein fraction, as commercially available from various sources, containing about 81% protein and between about 40 and about 300 mg sialic acid (SA) per gram of protein, but typically between about 40 and 60 mg SA/gm protein, and, thus, contributing between about 194 and about 1458 mg sialic acid per liter of formula, but typically between about 194 and 290 mg SA/liter of formula; or up to about 6 g/liter of a CGMP fraction having an enhanced level of sialic acid.

The casein glycomacropeptide that is useful in the present invention, in general, can be from any source and of any purity or grade that is suitable for inclusion in an infant formula. Casein glycomacropeptide may be extracted from milk using suitable processing. For example, the casein glycomacropeptide may be extracted from the retentate obtained from the concentration of whey protein. This may be done by at least partially removing lactose from the retentate and then adding ethanol to cause precipitation. The supernatant is then collected and dried to provide the casein glycomacropeptide. U.S. Pat. No. 5,216,129, which is incorporated herein entirely by reference to the extent that it does not conflict with information described herein, provides a more detailed description of this process. CGMP that is useful in the present method can also be produced according to the techniques described in U.S. Pat. Nos. 6,555,659, 5,280,107, 5,968,586, and 5,075,424, and in PCT/US94/15952, and WO 03/049547. Alternatively, the CGMP may be purchased from commercial sources such as, for example, The Tatua Co-Operative Dairy Company Limited, Tatuanui, Morrinsville, New Zealand, MD Foods Ingredients amba of DK-6920 Videbaek, Denmark or from DMV International of NCB-laan 80, NL-5460 BA Veghel, The Netherlands.

In the present method, it is preferred that the formula comprising casein glycomacropeptide is administered in an amount sufficient to provide 100 mg/kg-day of sialic acid to the mammal, and in some embodiments, the provision of 200 mg/kg-day of sialic acid to the mammal is more preferred.

In an embodiment of the present invention, it is preferred that the formula has total protein content of between 12 and 16 grams/liter of which no more than 40% by weight is provided by casein glycomacropeptide. It is more preferred that the formula comprises a total protein content of between 13 and 15 grams/liter of which no more than 30% by weight is provided by casein glycomacropeptide. In some embodiments, it is preferred that the protein in the formula comprises cow's milk protein, soy protein, or mixtures thereof.

Some embodiments of the present invention involve the use of novel CGMP products that contain levels of sialic acid that are higher than normally found in standard CGMP products that are commercially available. These novel products can be used alone or in combination to achieve sialic acid levels that mimic those found in breast milk, based on the sialic acid content of the various source ingredients. In one embodiment, the casein glycomacropeptide comprises a casein glycomacropeptide having an enhanced concentration of sialic acid.

As used herein, the terms “CGMP having an enhanced concentration of sialic acid” mean a casein glycomacropeptide (CGMP)-containing fraction of milk that has been treated to increase the level of sialic acid, and in which the level of sialic acid is higher, by any amount, than before the treatment. CGMP products with enhanced levels of sialic acid are described below in Reference Examples 2 and 3.

One such product, an example of which is described in Reference Example 2, can be referred to herein as “CGMP having an enhanced level of sialic acid”, or “high-sialic acid CGMP”. High-sialic acid CGMP has a sialic acid content of above about 60 mg/gm protein. It is preferred that the sialic acid content is above about 100 mg/gm protein, more preferred is above about 150 mg/gm protein, and yet more preferred is a sialic acid content of above 200 mg/gm protein. Typically, this product has a protein content of about 50%-60% by weight for a dry powder product, a sialic acid content of about 190-230 mg/gm protein, or about 100-130 mg/gm powder. In comparison, regular CGMP dry powder (for example, glycomacropeptide available from Tatua Co-Operative Dairy Company Limited) contains 81% protein by weight, and has a sialic acid content of about 52 mg/gm protein, or 42 mg/gm powder. It is apparent, therefore, that the sialic acid content of the high-sialic acid CGMP has been enhanced over that of the regular glycomacropeptide powder by about 3-fold on the basis of powder weight, and about 4-fold on the basis of protein content of the products. For comparison purposes, electrodialyzed (ED) whey powder contains about 14% protein on a dry basis, and contains about 30 mg of sialic acid/gm protein, or about 4.3 mg of sialic acid/gm of powder.

An advantage of using a high-sialic acid CGMP as a protein source in an infant formula is that the sialic acid content of the formula can be increased without replacing an undue amount of the conventional sources of protein that are used in the formula. This feature is useful in that it permits minimal disruption of the amino acid profile of the protein of the formula.

In a particular embodiment of a high-sialic acid CGMP, the product has a level of the amino acid threonine that is lower than the level of that amino acid in the glycomacropeptide from which the novel product is derived. As used herein, this type of high-sialic acid CGMP is referred to as “CGMP having an enhanced level of sialic acid and reduced threonine”, or “high-sialic acid CGMP with reduced threonine”. An example of this type of product is described below in Reference Example 3.

High-sialic acid CGMP with reduced threonine has a sialic acid content of above about 60 mg/gm protein and a threonine concentration that is lower than about 15 gm/16 gm nitrogen. It is preferred that the sialic acid content is above about 100 mg/gm protein, more preferred is above about 150 mg/gm protein, and yet more preferred is a sialic acid content of above 200 mg/gm protein Typically, high-sialic acid CGMP with reduced threonine can have a sialic acid content of from about 85 to about 150 mg sialic acid (SA)/ gram of powder, preferably from about 90 to about 140 mg SA/g powder, which is comparable to the sialic acid content of high-sialic acid CGMP. However, the threonine content of high-sialic acid CGMP with reduced threonine is only about one-fourth that of a commercial CGMP product. Preferably, the threonine content is below about 10 g/16 g nitrogen, more preferably below about 7 gm/16 gm nitrogen, even more preferably below about 5 g/16 g nitrogen, and yet more preferably below about 4 g/16 g nitrogen. Expressed in an alternative manner, the threonine content is below about 8% by weight of the total weight of amino acids of the protein, preferably below about 6%, more preferably below about 4%, and yet more preferably below about 3%.

An advantage provided by this type of enhanced sialic acid product is that in addition to the increase in sialic acid with reduced amino acid profile disruption, as discussed above, the threonine level of the protein sources in the infant formula can be controlled. This is desirable in some embodiments in order to reduce or eliminate the potential for hyperthreoninuria, or other disorder caused by, or exacerbated by, high levels of threonine in the diet.

By way of example, an infant formula that is useful in the present invention can be formulated to have a sialic acid content of at least 200 mg/liter and have a total protein content of between 12 and 16 grams/liter of which no more than 40% by weight is provided by a CGMP having an enhanced concentration of sialic acid. Preferably, such an infant formula has a total protein content of between 13 and 15 grams/liter of which no more than 30% by weight is provided by a CGMP having an enhanced concentration of sialic acid, more preferably, the infant formula has a total protein content of between 13 and 15 grams/liter of which no more than 15% by weight is provided by a CGMP having an enhanced concentration of sialic acid.

Also as an example, an infant formula that is useful in the present invention can be formulated to have a sialic acid content of at least 400 mg/liter and have a total protein content of between 13 and 15 grams/liter of which no more than 15% by weight is provided by a CGMP having an enhanced concentration of sialic acid.

The casein glycomacropeptide-supplemented formula that is useful in the present invention can be used in the same manner as any other commercial infant formula. It can be produced in powder form, for later reconstitution with a liquid, or it can be prepared in liquid form. The formula should be packaged, stored, handled, and distributed in the same manner as any other similar product, and should, in general, be used in the same fashion.

The following examples describe exemplary embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### REFERENCE EXAMPLE 1

This example illustrates the nutrient components in a commercial infant formula suitable for sialic acid addition for use in the present invention.

TABLE 1
Nutrient Information for Infant Formula (Enfamil ® Lipil with
Iron)

| NUTRIENTS | Per 100 Calories |
|---|---|
| (Normal Dilution) | (5 fl oz) |
| Protein, g | 2.1 |
| Fat, g | 5.3 |
| Carbohydrate, g | 10.9 |
| Water, g | 134 |
| Linoleic acid, mg | 860 |

Vitamins:

| | |
|---|---|
| A, IU | 300 |
| D, IU | 60 |
| E, IU | 2 |
| K, μg | 8 |
| Thiamin (Vitamin B1), μg | 80 |
| Riboflavin (Vitamin B2), μg | 140 |
| B6, μg | 60 |
| B12, μg | 0.3 |
| Niacin, μg | 1000 |
| Folic acid (Folacin), μg | 16 |
| Pantothenic acid, μg | 500 |
| Biotin, μg | 3 |
| C (Ascorbic acid), mg | 12 |
| Choline, mg | 12 |
| Inositol, mg | 6 |

Minerals:

| | |
|---|---|
| Calcium, mg | 78 |
| Phosphorus, mg | 53 |
| Magnesium, mg | 8 |
| Iron, mg | 1.8 |
| Zinc, mg | 1 |
| Manganese, μg | 15 |
| Copper, μg | 75 |
| Iodine, μg | 10 |
| Selenium, μg | 2.8 |
| Sodium, mg | 27 |
| Potassium, mg | 108 |
| Chloride, mg | 63 |

The ingredients of this particular formula are: reduced minerals whey, nonfat milk, vegetable oil (palm olein, soy, coconut, and high oleic sunflower oils), lactose, and less than 1%: mortierella alpina oil, crypthecodinium cohnii oil, vitamin A palmitate, vitamin D³, vitamin E acetate, vitamin K¹, thiamin hydrochloride, vitamin B6 hydrochloride, vitamin B¹², niacinamide, folic acid, calcium pantothenate, biotin, sodium ascorbate, inositol, calcium chloride, calcium phosphate, ferrous sulfate, zinc sulfate, manganese sulfate, cupric sulfate, sodium chloride, sodium citrate, potassium citrate, potassium hydroxide, sodium selenite, taurine, nucleotides (adenosine 5′-monophosphate, cytidine 5′-monophosphate, disodium guanosine 5′-monophosphate, disodium uridine 5′-monophosphate).

To use this particular formula to practice the present invention, it would be necessary to add, for example, casein glycomacropeptide to the formula in an amount sufficient to provide from about 250 mg per liter to about 1500 mg per liter of sialic acid to the composition described in Table 1. This added amount of sialic acid would be part of the total amount of protein (total protein of approximately 2.1 grams per 100 calories).

### EXAMPLE 1

This example illustrates a particular protein source combination for a total sialic acid content of approximately 250 mg per liter. The ingredients listed in Table 2 would be used to replace the protein component of the formula described in Table 1.

TABLE 2
Protein Source Composition A

| | mg | % of | | | |
|---|---|---|---|---|---|
| | SA/gm | protein in | g ingredient/ | g protein/ | mg |
| Ingredient | protein^{a} | ingredient | L | L | SA/L |
| Whey Protein | 23.00 | 35.00 | 20.26 | 7.09 | 163.08 |
| Concentrate | | | | | |
| Nonfat Dry | 6.37 | 34.00 | 15.38 | 5.23 | 33.31 |
| Milk, | | | | | |
| Low Heat | | | | | |
| CGMP^{b} | 52.00 | 81.00 | 1.45 | 1.17 | 61.07 |

Note:
^{a}“SA” in table means sialic acid.
^{b}CGMP means casein glycomacropeptide.

### EXAMPLE 2

This example illustrates a particular protein source combination for a total sialic acid content of approximately 360 mg per liter. The ingredients listed in Table 3 replace the protein component of the formula described in Table 1.

TABLE 3
Protein Source Composition B

| | mg | % of | | | |
|---|---|---|---|---|---|
| | SA/gm | protein in | g ingredient/ | g protein/ | mg |
| Ingredient | protein^{a} | ingredient | L | L | SA/L |
| Whey Protein | 23.00 | 35.00 | 37.00 | 12.95 | 297.85 |
| Concentrate | | | | | |
| CGMP^{b} | 52.00 | 81.00 | 1.45 | 1.17 | 61.07 |

Note:
^{a}“SA” in table means sialic acid.
^{b}CGMP means casein glycomacropeptide.

### EXAMPLE 3

This example illustrates a particular protein source combination for a total sialic acid content of approximately 600 mg per liters The ingredients listed in Table 4 replace the protein component of the formula described in Table 1.

TABLE 4
Protein Source Composition B

| | mg | % of | | | |
|---|---|---|---|---|---|
| | SA/gm | protein in | g ingredient/ | g protein/ | mg |
| Ingredient | protein^{a} | ingredient | L | L | SA/L |
| Whey Protein | 23.00 | 35.00 | 13.00 | 4.55 | 104.65 |
| Concentrate | | | | | |
| CGMP^{b} | 52.00 | 81.00 | 12.00 | 9.72 | 505.44 |

Note:
^{a}“SA” in table means sialic acid.
^{b}CGMP means casein glycomacropeptide.

### EXAMPLE 4

Table 5 illustrates an example of a complete nutritional formulation of an infant formula with a total sialic acid content of approximately 250 mg per liter.

TABLE 5
Exemplary infant formulation with sialic acid.

| | | | Amount per | |
|---|---|---|---|---|
| Ingredient | Weight | | 10000 liters | |
| Lactose (95% Solids) | | | 573.000 | kg |
| Fat Blend | | | 332.500 | kg |
| Whey Protein Concentrate (36% Protein, 5.8% Ash) | | | 202.578 | kg |
| Nonfat Milk Solid (36% Prot., 52% CHO) | | | 153.844 | kg |
| Casein glycomacropeptide (CGMP, | | | 14.500 | kg |
| 81.18% Prot.) | | | | |
| Mono-and Diglycerides | | | 7.233 | kg |
| Calcium Phosphate, Tribasic | | | 6.520 | kg |
| Single Cell Arachidonic Acid Oil | | | 6.485 | kg |
| Dry Vitamin Premix for Enfamil AR Liquid | | | 5.250 | kg |
| Ascorbic Acid | 2924.250 | g | | |
| Inositol | 834.750 | g | | |
| Corn Syrup Solid | 654.938 | g | | |
| Taurine | 582.750 | g | | |
| Niacinamide | 119.438 | g | | |
| Calcium Pantothenate | 44.730 | g | | |
| Vitamin B12, 0.1% in Starch | 29.400 | g | | |
| Biotin, 1% Trituration | 25.095 | g | | |
| Thiamine Hydroxhloride | 13.913 | g | | |
| Riboflavin | 10.238 | g | | |
| Pyridoxine Hydrochloride | 8.138 | g | | |
| Folic Acid | 2.363 | g | | |
| Lecithin Concentrate | | | 3.694 | kg |
| Potassium Citrate | | | 3.350 | kg |
| Single Cell Docosahexaenoic Acid Oil | | | 3.243 | kg |
| Nucleotide Premix for Enfamil Powder | | | 2.900 | kg |
| Maltodextrin, 15 DE | 2552.290 | g | | |
| Cytidine 5′-monophosphate, free | 202.710 | g | | |
| acid | | | | |
| Uridine 5′-monophosphate, | 59.740 | g | | |
| disodium salt | | | | |
| Adenosine 5′-monophosphate, free | 47.357 | g | | |
| acid | | | | |
| Guanosine 5′-monophosphate, | 37.903 | g | | |
| disodium salt | | | | |
| Carrageenan | | | 2.826 | kg |
| Magnesium Chloride | | | 1.657 | kg |
| Calcium Chloride, Dihydrate | | | 1.200 | kg |
| Choline Chloride | | | 0.700 | kg |
| Ferrous Sulfate Heptahydrate | | | 0.682 | kg |
| Sodium Citrate, Dihydrate, Granular | | | 0.455 | kg |
| Trace Mineral Premix w/Selenite | | | 0.392 | kg |
| Trituration | | | | |
| Zinc Sulfate, Monohydate | 276.238 | g | | |
| Sodium Selenite Trituration, 0.5% | 65.907 | g | | |
| Cupric Sulfate, powder | 29.510 | g | | |
| Lactose, Grind A | 16.323 | g | | |
| Manganese Sulfate, monohydrate | 4.022 | g | | |
| Vitamin A, D, E, K Premix, Enfamil Liquid | | | 0.324 | kg |
| Tocopherol Acetate | 160.882 | g | | |
| Soybean Oil | 139.612 | g | | |
| Vitamin A Palmitate | 17.253 | g | | |
| Cholecalciferol Concentrate | 5.715 | g | | |
| Vitamin K1, Liquid | 0.538 | g | | |
| Ascorbic Acid | | | 0.150 | kg |
| L-Carnitine | | | 0.150 | kg |
| Water, Defluoridated, q.s. to | | | 10310.986 | kg |

| | |
|---|---|
| Potassium Hydroxide | — |

Table 6 and Table 7 show the content of specific components of the formulation described in Table 5 as a percentage of 1) weight to weight, 2) weight to volume, and 3) calories. The specific gravity of this particular formulation is 1.0310986.

TABLE 6
Infant formulation composition.

| Component | % w/w | % w/v |
|---|---|---|
| Protein | 1.38 | 1.42 |
| Fat | 3.50 | 3.61 |
| Carbohydrate | 7.20 | 7.43 |
| Ash | 0.37 | 0.38 |
| Total Solids | 12.45 | 12.84 |

TABLE 7
Infant formula caloric distribution

| Component | % |
|---|---|
| Protein | 8.38 |
| Fat | 47.83 |
| Carbohydrate | 43.79 |

### EXAMPLE 5

Table 8 illustrates the nutritional content of the formulation presented in Example 4 per 100 calories, as well as per 100 milliliters of formula.

TABLE 8
Nutritional content of infant formulation.

| | Per 100 Cal | Per 100 ml |
|---|---|---|
| Calories, Cal | 100 | 68 |
| Protein, g | 2.1 | 1.42 |
| Fat, g | 5.3 | 3.6 |
| Carbohydrate, g | 10.9 | 7.4 |
| Linoleic Acid, mg | 860 | 580 |
| Linolenic Acid, mg | 80 | 54 |
| Arachidonic Acid, mg | 34 | 23 |
| Docosahexaenoic Acid, | 17 | 11.5 |
| mg | | |
| Vitamin A, IU | 300 | 200 |
| Vitamin D, IU | 60 | 41 |
| Vitamin E, IU | 2 | 1.35 |
| Vitamin K1, mcg | 12 | 8.1 |
| Thiamin, mcg | 120 | 81 |
| Riboflavin, mcg | 140 | 95 |
| Vitamin B6, mcg | 60 | 41 |
| Vitamin B12, mcg | 0.5 | 0.3 |
| Niacin, mcg | 1200 | 812 |
| Folic Acid, mcg | 16 | 10.8 |
| Pantothenic Acid, mcg | 500 | 340 |
| Biotin, mcg | 3 | 2 |
| Vitamin C, mg | 14 | 9.5 |
| Choline, mg | 12 | 8.1 |
| Inositol, mg | 6 | 4.1 |
| Taurine, mg | 6 | 4.1 |
| L-Carnitine, mg | 2 | 1.35 |
| Calcium, mg | 78 | 53 |
| Phosphorus, mg | 53 | 36 |
| Magnesium, mg | 8 | 5.4 |
| Iron, mg | 1.8 | 1.2 |
| Zinc, mg | 1 | 0.68 |
| Manganese, mcg | 26 | 17.6 |
| Copper, mcg | 85 | 57 |
| Iodine, mcg | 15 | 10 |
| Sodium, mg | 27 | 18.3 |
| Potassium, mg | 108 | 73 |
| Chloride, mg | 63 | 43 |
| Selenium, mcg | 2.8 | 1.89 |
| Sialic acid, mg | 37 | 25 |
| Calcium/Phosphorus Ratio | — | — |
| AMP Equivalents, mg (a) | 0.5 | 0.34 |
| CMP Equivalents, mg (a) | 2.5 | 1.69 |
| GMP Equivalents, mg (a) | 0.3 | 0.20 |
| UMP Equivalents, mg (a) | 0.9 | 0.61 |
| Nucleotide Equivalents, | 4.2 | 2.84 |
| mg (a) | | |
| TPAN-AMP, mg | — | — |
| TPAN-CMP, mg | — | — |
| TPAN-GMP, mg | — | — |
| TPAN-UMP, mg | — | — |
| Total TPAN, mg | — | — |
| TPAN-CMP/TPAN-GMP | — | — |
| Ratio | | |

Note:
Sum of the nucleotide and corresponding nucleoside expressed as the nucleotide weights.

### REFERENCE EXAMPLE 2

This illustrates the production of a CGMP product having enhanced levels of sialic acid.

A fraction of cheese whey that is enriched in GMP is fractionated by using anion chromatography to yield a fraction that is enhanced in sialic acid. This product exhibits an amino acid profile similar to that of currently commercially available GMP (available from Tatua Co-Operative Dairy Company Limited, Tatuanui, Morrinsville, New Zealand), but contains from 1.5-3 times the sialic acid content of currently available GMP products.

The sialic acid-enhanced fraction can be desalted, if desired, by electrodialysis, for example, and can be dried to yield a dry powder product, which is then useable for introduction into a liquid or a dry infant formula mix. This product is a high-sialic acid CGMP and is available as of the filing date of the present application from Tatua Co-Operative Dairy Company Limited, as products designated as X4738, X4739, X4740, and X4741. The protein content, sialic acid content, and amino acid profile of those materials is described in Table 9.

TABLE 9
Amino acid profile and sialic acid content of four examples
of high-sialic acid CGMP products.

| | High-Sialic Acid CGMP Product | | | | | | |
|---|---|---|---|---|---|---|---|
| | Samples | | | | | CGMP | ED |
| Amino Acid | X4738 | X4739 | X4740 | X4741 | Average | Powder | Whey Powder |
| Arginine | 1.22 | 0.96 | 0.69 | 0.7 | 0.89 | 1.4 | |
| Histidine | 0.76 | 0.7 | 0.59 | 0.59 | 0.66 | 1 | |
| Isoleucine | 10.36 | 8.42 | 11.28 | 11.51 | 10.39 | 11.6 | |
| Leucine | 4 | 3.36 | 3.19 | 3.21 | 3.44 | 4.6 | |
| Lysine | 7.48 | 7.91 | 6.89 | 7.08 | 7.34 | 8.3 | |
| Methionine | 2 | 2.04 | 1.63 | 1.63 | 1.83 | 1.5 | |
| Cystine | 0.21 | 0.47 | 0.13 | 0.09 | 0.23 | 0.2 | |
| phenylalanine | 1.54 | 5.88 | 1.96 | 2.08 | 2.87 | 1.8 | |
| Tyrosine | 0.35 | 0.17 | 0.1 | 0.09 | 0.18 | 0.4 | |
| Threonine | 13.13 | 15.16 | 17.18 | 17.57 | 15.76 | 15.9 | |
| Tryptophan | 0 | 0 | 0 | 0 | 0.00 | 0 | |
| Valine | 8.69 | 7.55 | 9.38 | 9.51 | 8.78 | 9.7 | |
| Alanine | 6.8 | 6.58 | 6.41 | 6.53 | 6.58 | 6.6 | |
| aspartic acid | 10.61 | 12.12 | 9.93 | 10 | 10.67 | 11.1 | |
| glutamic acid | 22.91 | 24.23 | 23.28 | 23.14 | 23.39 | 26.2 | |
| Glycine | 1.37 | 1.46 | 1.31 | 1.34 | 1.37 | 1.5 | |
| Proline | 11.13 | 10.19 | 10.78 | 9.57 | 10.42 | 14.2 | |
| Serine | 8.14 | 9.66 | 8.74 | 9.06 | 8.90 | 8.1 | |
| TOTAL | 110.7 | 116.86 | 113.47 | 113.7 | 113.68 | 124.1 | |
| % protein | 51.88 | 49.92 | 57.87 | 60.05 | 54.93 | 81 | 14.31 |
| mgSA/gm | 188.43 | 227.25 | 224.83 | 215.68 | 214.05 | 52 | 29.92 |
| protein | | | | | | | |
| mgSA/gm | 97.76 | 113.44 | 130.11 | 129.52 | 117.71 | 42.12 | 4.28 |
| powder | | | | | | | |
| Average | | | | | | | |

Amino acid levels are expressed as grams of the amino acid per 16 grams of nitrogen.
CGMP Powder is commercial glycomacropeptide from Tatua Co-Operative Dairy Company Ltd.
ED Whey Powder is commercial electrodialyzed whey powder
Samples X4738-X4741 are samples of high-sialic acid CGMP available from Tatua Co-Operative Dairy Company Limited, Tatuanui, Morrinsville, New Zealand.

### REFERENCE EXAMPLE 3

This illustrates the production of a CGMP product having enhanced levels of sialic acid and low levels of threonine.

A fraction of cheese whey that is enriched in GMP is subjected to a partial proteolytic hydrolysis followed by fractionation by using anion chromatography to yield a fraction that is enhanced in sialic acid and has a low threonine content. This product contains from 1.5-3 times the sialic acid content of currently available GMP products, but the level of threonine is reduced to about one-fourth that of the starting GMP material.

The sialic acid-enhanced, low threonine fraction can be desalted, if desired, by electrodialysis, for example, and can be dried to yield a dry powder product, which is then useable for introduction into a liquid or a dry infant formula mix. This product is a high-sialic acid CGMP with reduced threonine and is available as of the filing date of the present application from Tatua Co-Operative Dairy Company Limited, Tatuanui, Morrisnville, New Zealand, as product designated as W4733. The protein content, sialic acid content, and amino acid profile of that material is described in Table 10.

TABLE 10
Amino acid profile and sialic acid content of high-sialic
acid CGMP with reduced threonine.

| | High-Sialic Acid CGMP with | | |
|---|---|---|---|
| | Reduced Threonine | CGMP | ED Whey |
| Amino Acid | W4733 | Powder | Powder |
| arginine | 2.3 | 1.4 | |
| histidine | 0 | 1 | |
| isoleucine | 13.1 | 11.6 | |
| leucine | 5.3 | 4.6 | |
| lysine | 3.2 | 8.3 | |
| methionine | 0.7 | 1.5 | |
| cystine | 0.1 | 0.2 | |
| phenylalanine | 0 | 1.8 | |
| tyrosine | 0 | 0.4 | |
| threonine | 3.8 | 15.9 | |
| tryptophan | 0 | 0 | |
| valine | 16.3 | 9.7 | |
| alanine | 15.9 | 6.6 | |
| aspartic acid | 6.3 | 11.1 | |
| glutamic acid | 38.9 | 26.2 | |
| glycine | 2.5 | 1.5 | |
| proline | 16.9 | 14.2 | |
| serine | 0 | 8.1 | |
| TOTAL | 125.4 | 124.1 | |
| % protein | | 81 | 14.31 |
| mgSA/gm | | 52 | 29.92 |
| protein | | | |
| mgSA/gm | 138.03 | 42.12 | 4.28 |
| powder | | | |
| (Repeat | 117.02 | | |
| analysis) | | | |
| Average | 127.525 | | |

Amino acid levels are expressed as grams of the amino acid per 16 grams of nitrogen
CGMP Powder is commercial glycomacropeptide from Tatua Co-Operative Dairy Company Ltd.
ED Whey Powder is commercial electrodialyzed whey powder
Samples W4731, W4733, and W4735 are samples of enhanced sialic acid CGMP with reduced threonine available from Tatua Co-Operative Dairy Company Limited, Tatuanui, Morrinsville, New Zealand.

It is noted that the threonine level of the novel product are about one-fourth that of commercial CGMP. Accordingly, it is believed that use of a high-sialic acid CGMP with reduced threonine in an infant formula can provide a formula having a high level of sialic acid at normal, desirable protein levels of about 14 g protein/liter, and yet provide a desirable amino acid profile and low levels of threonine.

### EXAMPLE 6

This illustrates the use of a CGMP fraction having enhanced levels of sialic acid in an infant formula.

The CGMP product having enhanced levels of sialic acid can be used as a protein source in an infant formula in the same manner as a whey powder or normal CGMP powder. By way of example, Table 11 shows the sialic acid content that could be expected for an infant formula in which the protein content is supplied by conventional sources. The amount of CGMP powder that is used is limited in order to avoid undue deviation of the amino acid profile of the protein that is provided from a desirable infant standard profile.

TABLE 11
Sialic acid content of infant formula with protein provided
by conventional sources:

| | mg sialic acid/ | Percent | grams | mg sialic |
|---|---|---|---|---|
| Protein Source | gm protein | protein | protein/liter | acid/liter |
| Whey protein | 23 | 35% | 6.82 | 156.77 |
| concentrate | | | | |
| Nonfat dry milk, | 6.37 | 34% | 6.25 | 39.8 |
| low heat | | | | |
| CGMP Powder | 52 | 81% | 1.11 | 57.62 |
| Total | | | 14.17 | 254.18 |

The CGMP powder that is used in this formulation can be replaced by novel CGMP product having an enhanced level of sialic acid, as is described above in Reference Example 3. Table 12 shows that when this is done, the sialic acid content of the formulation is more than doubled with no further disruption of the amino acid profile of the protein.

TABLE 12
Sialic acid content of infant formula with protein provided
by conventional sources plus a CGMP product having an enhanced level
of sialic acid:

| | mg sialic acid/ | Percent | grams | mg sialic |
|---|---|---|---|---|
| Protein Source | gm protein | protein | protein/liter | acid/liter |
| Whey protein | 23 | 35% | 6.82 | 156.77 |
| concentrate | | | | |
| Nonfat dry milk, | 6.37 | 34% | 6.25 | 39.8 |
| low heat | | | | |
| CGMP Product | 214 | 54.93% | 1.11 | 237.54 |
| with enhanced | | | | |
| sialic acid | | | | |
| Total | | | 14.17 | 434.12 |

If the CGMP powder having enhanced sialic acid levels were to be used at double the levels described above at the expense of nonfat dry milk, the sialic acid content of the formula could be increased to the level shown in Table 13.

TABLE 13
Sialic acid content of infant formula with protein provided
by conventional sources plus a CGMP product with an enhanced level of
sialic acid:

| | mg sialic acid/ | Percent | grams | mg sialic |
|---|---|---|---|---|
| Protein Source | gm protein | protein | protein/liter | acid/liter |
| Whey protein | 23 | 35% | 6.82 | 156.77 |
| concentrate | | | | |
| Nonfat dry milk, | 6.37 | 34% | 5.14 | 32.74 |
| low heat | | | | |
| CGMP Product | 214 | 54.93% | 2.22 | 475.08 |
| with enhanced | | | | |
| sialic acid | | | | |
| Total | | | 14.18 | 664.59 |

### EXAMPLE 7

This example illustrates the efficacy of dietary supplementation with casein glycomacropeptide in increasing the salivary sialic acid content of piglets.

Twenty 3-day-old male domestic piglets (Sus scorfa) from 4 litters were distributed evenly to 2 groups, balanced for weight and litter group. The control group (n=10) was fed a standard diet of soy/whey/casein sow milk pig-replacer (55:9:36) containing 150 mg/L of naturally-occurring sialic acid. The treatment group (n=10) received a similar formula in which casein glycomacropeptide replaced some of the casein such that the final level of sialic acid was 600 mg/L. Milk intake in both groups was 285 ml/kg/day during the first 2 weeks and 230 ml/kg/day for the remaining weeks. On a body weight basis, the control group therefore received 43 mg/kg/day of sialic acid and the treatment group 170 mg/kg/day. Saliva samples (0.5-1 mL) were collected once per week using a sterile plastic pipette immediately before the morning feed. Free and bound sialic acid contents were determined using high performance liquid chromatography. During the first two weeks, 7 piglets in the control group and 4 piglets in the treatment group required antibiotic medication for weaning diarrhea.

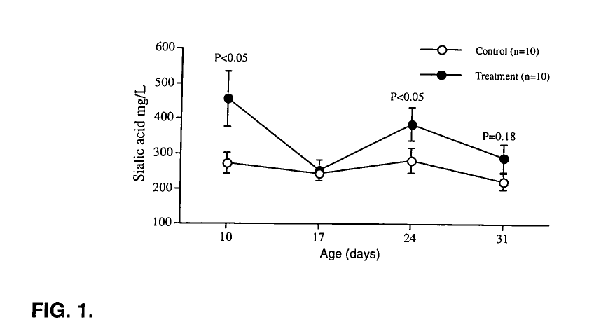

Rate of weight gain was similar in the two groups (170 g/day in the control group, 155 g/day in treatment P=0.44). Total salivary sialic acid content varied from 2% to 41% higher in the treatment group compared with the controls (FIG. 1.). The difference was significant on day 10 and day 24 (P<0.05), but not on days 17 or 31. Although there was a sharp decline in the salivary sialic acid content for the treatment group between day 10 and day 17, time trends over the whole time period were not significantly different (P>0.05). The majority of sialic acid was in the bound form (>93%) and showed the same trends.

The findings of this study show that a protein-bound dietary source of sialic acid such as casein glycomacropeptide increases the sialic acid content of saliva.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions by those of ordinary skill in the art without departing from the scope of the invention, it is intended that all matter contained in the above description and shown in the accompanying drawings shall be interpreted as illustrative and not in a limiting sense. In addition it should be understood that aspects of the various embodiments may be interchanged both in whole or in part.

What is claimed is:

1. A method of increasing the salivary sialic acid content in a mammal that is in need of increasing salivary sialic acid content, the method comprising administering to the mammal casein glycomacropeptide in an amount sufficient to increase the salivary sialic acid content in the mammal.

2. The method according to claim 1, wherein the mammal is between about 1 day and about 4 years of age.

3. The method according to claim 1, wherein the mammal is a neonatal mammal.

4. The method according to claim 1, wherein the mammal that is in need of increasing salivary sialic acid content is a mammal in which at least a portion of its nutritional requirement has been supplied by a formula having less than about 100 mg/L of sialic acid.

5. The method according to claim 4, wherein the mammal that is in need of increasing salivary sialic acid content is a mammal in which substantially all of its nutritional requirement has been supplied by administration of a formula having less than about 100 mg/L of sialic acid.

6. The method according to claim 5, wherein a major part of the protein contained in the formula is soy protein or cow's milk protein.

7. The method according to claim 6, wherein at least about 75% by weight of the protein contained in the formula is soy protein or cow's milk protein.

8. The method according to claim 6, wherein substantially all of the protein contained in the formula is soy protein or cow's milk protein.

9. The method according to claim 1, wherein the mammal that is in need of increasing salivary sialic acid content is a mammal in which its nutritional requirement has been supplied by a diet providing sialic acid in an amount that is lower than would normally be obtained from breastfeeding.

10. The method according to claim 9, wherein the diet providing a lower amount of sialic acid than would normally be obtained from breastfeeding comprises a liquid formula having a sialic acid content that is below about 100 mg/L.

11. The method according to claim 9, wherein the diet providing a lower amount of sialic acid than would normally be obtained from breastfeeding comprises a liquid formula having a sialic acid content that is below about 200 mg/L.

12. The method according to claim 1, wherein administering the formula comprises enteral administration.

13. The method according to claim 1, wherein the formula is an infant formula.

14. The method according to claim 1, wherein the formula is a nutritionally complete infant formula comprising carbohydrate, lipid, and protein.

15. The method according to claim 14, wherein the protein comprises a material that is selected from cow's milk protein, soy protein, or a mixture thereof.

16. The method according to claim 1, wherein the formula comprising casein glycomacropeptide is administered in an amount sufficient to provide at least about 100 mg/kg-day of sialic acid to the mammal.

17. The method according to claim 1, wherein the formula comprising casein glycomacropeptide is administered in an amount sufficient to provide at least about 200 mg/kg-day of sialic acid to the mammal.

18. The method according to claim 1, wherein the formula comprises a liquid having a sialic acid concentration of at least about 200 mg/liter.

19. The method according to claim 18, having a sialic acid level of at least about 300 mg/liter.

20. The method according to claim 18, having a sialic acid level of at least about 600 mg/liter.

21. The method according to claim 1, comprising a formula having a total protein content of between 12 and 16 grams/liter of which no more than 40% by weight is provided by casein glycomacropeptide.

22. The method according to claim 21, wherein the formula comprises a total protein content of between 13 and 15 grams/liter of which no more than 30% by weight is provided by casein glycomacropeptide.

23. The method according to claim 1, wherein the casein glycomacropeptide comprises a casein glycomacropeptide having an enhanced concentration of sialic acid.

24. The method according to claim 23, wherein the casein glycomacropeptide having an enhanced concentration of sialic acid comprises high-sialic acid casein glycomacropeptide with reduced threonine.

25. The method according to claim 24, wherein the formula has a total protein content of between 13 and 15 grams/liter of which no more than 30% by weight is provided by a casein glycomacropeptide having an enhanced concentration of sialic acid and having a threonine content of not over 10 grams/16 grams nitrogen.

26. The method according to claim 1, wherein the formula has a total protein content of about 14 grams/liter of which not over 4% by weight is threonine and having a sialic acid content of at least 400 mg/liter.

27. The method according to claim 1, further comprising measuring the salivary sialic acid content of the mammal following the administration of the casein glycomacropeptide.

28. A method of increasing salivary sialic acid content in a mammal, the method comprising:
determining whether the mammal is one that is in need of increasing salivary sialic acid content; and, if so
administering to the mammal a formula comprising casein glycomacropeptide in an amount sufficient to increase salivary sialic acid content in the mammal.
